(19) United States
(12) Patent Application Publication
(10) Pub. No.: US 2007/0238950 A1
(43) Pub. Date: Oct. 11, 2007

(54) Separate and combined multi-modality diagnostic imaging system

(76) Inventors: A. Hans Vija, Evanston, IL (US); Guenter Hahn, Hoffman Estates, IL (US); Steve D. Quam, Carpentersville, IL (US); Markus Lusser, Cary, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/388,894

(22) Filed: Mar. 24, 2006

(51) Int. Cl. A61B 5/05

(52) U.S. Cl. 600/407

(57) ABSTRACT

A multi-modality diagnostic imaging system includes a first imaging subsystem, such as a computed tomographic (CT) system, for performing a first imaging procedure on a subject. The first imaging system has a gantry that slides along rails. A second imaging subsystem, such as a nuclear medicine system (NUC), performs a second imaging procedure on a subject. The second imaging subsystem is separate from the first imaging system, but in the same room. The second imaging subsystem has a gantry or is gantryless. A patient couch supports a subject. The patient couch is supported on a couch support having vertical adjustment and horizontally fixed. In this manner, the patient is maintained in the same position for multiple types of scans using the same couch support without horizontal translation of the patient couch and patient.

### BACKGROUND OF THE INVENTION

1. Field of the Invention

The present invention relates to the art of diagnostic imaging. It finds particular application in conjunction with a separate and combined multi-modality diagnostic imaging system, and more particularly, a separate and combined computed tomographic/nuclear medicine (CT/NUC) diagnostic imaging system.

2. Description of the Background Art

Nuclear medicine imaging, such as single photon emission computed tomography (SPECT), is used to study radionuclide distribution in subjects. Typically, one or more radiopharmaceuticals are injected into a subject. The radiopharmaceuticals are commonly injected into the subject's blood stream for imaging the circulatory system or for imaging specific organs which absorb the injected radiopharmaceuticals. One or more gamma or scintillation camera heads are placed closely adjacent to a surface of the subject to monitor and record emitted radiation.

In single photon-emission computed tomography, the camera head(s) is typically rotated or indexed around the subject to collect the emitted radiation from a plurality of directions. The radiation data from the multiplicity of directions monitored over several minutes is reconstructed into a three dimensional image representation of the radiopharmaceutical distribution within the subject.

In computed tomographic (CT) diagnostic imaging, a thin fan-shaped beam of radiation is projected from an x-ray source through a region of interest. The radiation source is rotated rapidly about the region of interest such that the same thin slice of the region of interest is irradiated from a multiplicity of directions spanning 360 degrees. For a volumetric image representation, the source rotates at speeds on the order of 1 sec/revolution or less while the patient or the CT gantry is moved longitudinally to irradiate the patient along a spiral path.

Typically, computed tomographic (CT) imaging systems and nuclear medicine imaging systems are located in separate imaging suites with no physical and/or functional connections therebetween. The diagnostic images that result from the respective imaging studies can be viewed concurrently on adjoining screens for diagnostic purposes. However, the value of these image combinations and comparisons is compromised by having been obtained in separate study episodes. These separate study episodes are performed at different locations between which the patient typically walks. Repositioning the patient in the same position is imprecise. The episodes are usually separated by significant time intervals (days or even weeks) after which significant functional and anatomical changes can occur in addition to the repositioning problem. These separate study episodes are also performed by different medical personnel which distracts from the comparative value of the separate images.

U.S. Pat. No. 5,391,877 describes a dedicated combined diagnostic, imaging device that fuses together data obtained by a computed tomographic (CT) scanner and a single photon emission computed tomographic scanner (SPECT) to yield a color shaded relief image. The combined diagnostic imaging device includes combined gantries supporting both of the CT and SPECT scanners, a computer, a printer, and a table top that passes through both gantries while holding a patient in a fixed position on the table top.

U.S. Pat. No. 6,754,520 describes a system and method for handling a patient in a tomographic imaging system using a plurality of imaging devices. The imaging devices each have a bore through which a patient is translated during scanning. One or more patient support structures extend from the front of the tomographic imaging system, where the patient is initially placed, through the bores of the system. The patient is translated through the bores of the system and along the patient support structures by an actuator.

U.S. Pat. No. 6,603,991 describes a multi-modality diagnostic imaging system with at least two imaging devices with one of such devices having a stationary gantry. The patient is placed on a patient bed and the bed is moved from one imaging device the other to perform the imaging scans on the patient. If the imaging device has a gantry, the bed is translated through the bore during the scan.

U.S. Pat. No. 6,205,347 describes a multi-modality diagnostic imaging system having a patient transfer subsystem that transfers a patient couch between two imaging subsystems. The two imaging subsystems can be operated concurrently to perform different imaging procedures on different subjects supported by separate patient couches.

However, the patient in each of the foregoing systems is horizontally moved from system to system to perform multiple scans, and for imaging devices with a bore, the patient is further horizontally moved or translated through the bore during the scan. This horizontal movement of the patient causes patient anxiety and may inadvertently change the placement of the patient and their internal organs from scan to scan causing difficulties in the registration of the various scans for comparison and/or combination. Further, combination systems, for example, as that disclosed in U.S. Pat. No. 5,391,877, make access to the different imaging subsystems and maintenance thereof difficult.

Accordingly, it has been considered desirable to develop a new and improved separate and combined multi-modality diagnostic imaging system which meets the above-stated needs and overcomes the foregoing difficulties and others while providing better and more advantageous results.

### SUMMARY OF THE INVENTION

A new and improved separate and combined multi-modality medical imaging system and diagnostic suite containing same is provided which meet the above-stated needs and overcomes the foregoing difficulties and others while providing better and more advantageous results.

In accordance with one aspect of the present invention, a multi-modality medical imaging apparatus is disclosed. The multi-modality medical imaging apparatus includes a first imaging device for obtaining one or more tomographic images of a subject patient, at least a portion of the first imaging device having a first bore. A second imaging device is provided for obtaining one or more tomographic images of a subject patient, at least a portion of the second imaging device having an imaging section. A patient pallet adapted for supporting a patient subject. A horizontally fixed patient support structure supports the patient pallet and has vertical adjustment to position the patient pallet in substantial alignment with the first bore of the first imaging device. A first positioning device translates the first imaging device axially along the patient pallet in forward and reverse directions during formation of one or more images by the first imaging device. A second positioning device positions the imaging section of the second imaging device relative to the patient support during formation of one or more images by the second imaging device. In another embodiment, a third medical imaging device is provided. The second or third medical device can have a sliding gantry. However, all three medical devices and the pallet support structure are located in the same room.

The various imaging devices are selected from the first group consisting of CT, MRI, X-Ray, and Ultrasound devices and the second group consisting of SPECT and PET devices. When there are two imaging devices and the first imaging device is selected from the first group, the second imaging device can be a different one from the first group or one from the second group. When there are three imaging devices, the first two are selected from the first group and the third from the second group.

In accordance with another aspect of the present invention, a diagnostic imaging suite is disclosed. The diagnostic imaging suite includes a first medical imaging device positioned within the imaging suite for performing a first imaging procedure on a subject supported on a patient pallet, wherein the first medical imaging device has a first sliding gantry. A second medical imaging device is also positioned within the imaging suite separate from the first medical imaging device for performing a second imaging procedure on a subject supported on the patient pallet. A patient pallet support for the patient pallet has vertical adjustment. A pair of rails are positioned parallel to the longitudinal axis of the patient pallet support, with each of the pair of rails positioned on either side of the patient pallet support for use with the first sliding gantry. The second imaging system is positionable in a non-use position and in an in-use position. In the non-use or rest position the second imaging system does not interfere with the first imaging system when in it is in use. In one embodiment, the second image device also has a sliding gantry. In such a configuration, the gantries of the first and second imaging devices can use the same or different rails.

In accordance with another aspect of the present invention, a patient table for use in combination with a medical imagining apparatus having a first imaging device with a first bore and a second imaging device with a second bore is disclosed. The patient table includes a patient support structure adapted to extend through the first and second bores of the first and second imaging devices. The patient support structure has a first end with an first end support that is vertically adjustable and a second end with a second end support that is vertically adjustable. The first and second end supports move the patient support structure vertically up and down to substantially align the patient support structure with the respective bore of the first or second imaging device in an installed position. A pallet for supporting a patient on the patient support structure is located between the first and second bores of the first and second imaging devices in an installed position.

One advantage of the present invention is the provision of a new and improved separate and combined multi-modality diagnostic imaging system that provides CT anatomical imaging with nuclear medicine functional imaging in one clinical study episode (location and time period) and in such a manner that clinical productivity and patient care are improved. Further, patient anxiety is reduced since they are moved during the scanning episodes.

Another advantage of the present invention is the provision of a new and improved separate and combined multi-modality diagnostic imaging system where a patient is imaged on the exact same table, and in a single imaging episode. This permits the subject to be in the same external and internal imaging states during both imaging studies.

Yet another advantage of the present invention is the provision of a new and improved separate and combined multi-modality diagnostic imaging system that allows the combination functional images (i.e. nuclear) with anatomical images (i.e. CT) via image fusion and side by side image registration since various scans are performed in a single episode.

Still further advantages of the present invention will become apparent to those of ordinary skill in the art upon reading and understanding the following detailed description of the preferred embodiments.

### BRIEF DESCRIPTION OF THE DRAWINGS

The invention may take form in various components and arrangements of components, and in various steps and arrangements of steps. The drawings are only for purposes of illustrating a preferred embodiment(s) and are not to be construed as limiting the invention. The present invention is more fully described in the following attached drawings in which corresponding elements are designated by like reference numbers:

FIG. 1 is a perspective schematic view of a separate and combined multi-modality medical imaging system that incorporates a computed tomographic (CT) scanner system with a sliding gantry and nuclear medicine (NUC) system, specifically a SPECT device, mounted to the ceiling in accordance with a first embodiment of the present invention;

FIG. 2 is a diagram schematically showing the overall configuration of a multi-modality medical imaging system that incorporates a computed tomographic (CT) scanner system with a sliding gantry and an X-ray fluoroscopy device mounted to the ceiling in accordance with a second embodiment of the present invention;

FIG. 3 is a pictorial representation of a diagnostic suite 300 showing the medical imaging apparatus 100 of FIG. 1;

FIG. 4 is a perspective schematic view of a separate and combined multi-modality medical imaging system that incorporates a computed tomographic (CT) scanner system with a sliding gantry, an MRI with a sliding gantry, and a SPECT device mounted to the ceiling in accordance with another embodiment of the present invention;

FIG. 5 is a plan schematic view of a separate and combined multi-modality medical imaging system that incorporates a computed tomographic (CT) scanner system with a sliding gantry, an MRI with a sliding gantry, and a SPECT device mounted to the ceiling similar to that shown in FIG. 4, except that a different embodiment of a pallet support is used in accordance with another embodiment of the present invention; and

FIG. 6 is an elevational view of the combined multi-modality medical imaging system of FIG. 5.

### DETAILED DESCRIPTION OF THE PREFERRED EMBODIMENT(S)

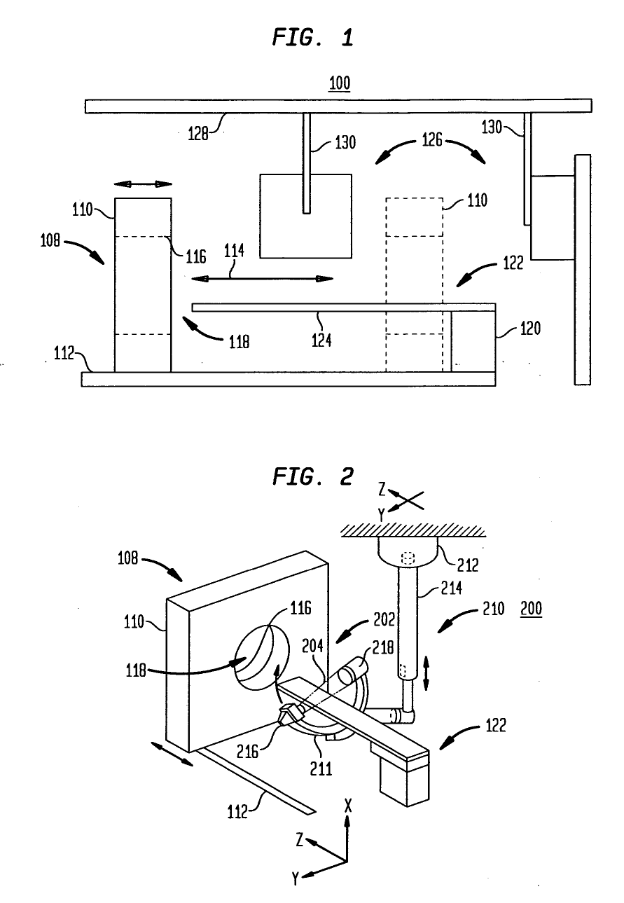

With reference to FIG. 1, a first embodiment of a separate and combined multi-modality medical imaging system 100 according to the present invention is shown. The imaging system has a computed tomographic (CT) scanner 108 with a non-rotating, sliding gantry 110 mounted on tracks 112 that extend parallel to the longitudinal axis 114. This allows the gantry 110 to be moved parallel to the longitudinal axis 114 and placed at the desired location during data collection. An x-ray tube (not shown) is rotatably mounted on a rotating gantry (not shown). The gantry 110 includes a cylinder or bore 116 that defines a patient examination region 118. An array of radiation detectors (not shown) is disposed concentrically around the patient receiving region 118. The x-ray detectors can mounted on the gantry 110 such that an arc segment of the detectors receives radiation from the x-ray tube (not shown) which has traversed the examination region 118. Alternatively, an arc segment of radiation detectors can be mounted to the rotating gantry (not shown) to rotate with the x-ray tube (not shown). The CT system 108 includes conventional pilot, axial, and spiral scanning and imaging capability. Details regarding the x-ray tube and detectors are within the skill of the art, for example, see U.S. Pat. No. 6,205,347. Similarly, information regarding the sliding gantry and its components are also within the skill of the art, for example, as shown in U.S. Pat. No. 6,205,347, which discloses drive motors, drive wheels attached thereto, and the use of rails or tracks.

The sliding gantry 110 is shown in a non-use or rest position in FIG. 1. The dotted outline of the sliding gantry 110 represents the fully extended or stop position near to the base 120 of the patient pallet support structure 122. One end portion of a cantilevered pallet support 124 is attached to the base 120.

The SPECT detectors 126 are slidably attached to the ceiling with tracks 128 and supports 130. The detectors 126 are shown in their stored position facing away from radiation towards the walls of the room.

FIG. 2 is a diagram schematically showing another embodiment of the multi-modality system of the present invention having an X-ray CT scanner with a sliding gantry and X-ray fluoroscopy device.

A multi-modality system 200 shown in FIG. 2 comprises an X-ray fluoroscopy device 202, a sliding X-ray CT scanner 108 on a pair of rails 112, and a patient pallet support structure 122. The console, monitor, and input device (not shown) are components that can optionally be shared by the fluoroscopy apparatus 202 and CT scanner 108. The patient pallet support structure 122 supports the cantilevered pallet support 124 and can be adjusted vertically, but is otherwise horizontally fixed. A patient can lie down on the pallet support 124. The X-ray fluoroscopy device 202 is positioned out of the path of the sliding CT scanner 108, when it is in use. The X-ray fluoroscopy apparatus 202 in use is positioned such that the pallet support 124 is placed within its detection zone 204. In the meantime, the CT scanner 108 is slid to its non-use or rest position as shown in FIG. 2 so as not to interfere with the X-ray fluoroscopy device 202.

In the X-ray fluoroscopy device 202, the support 210 is capable of traveling the entire C-arm 211 at least three-dimensionally in the X-, Y- and Z-directions. (In the orthogonal coordinate, the Z-direction corresponds to the longitudinal direction of the pallet support 124.) The support 210 includes a ceiling-attaching portion 212 enabling two-dimensional travel in the Y-Z plane parallel to the ceiling and a pillar portion 214 enabling one-dimension travel along the X(height)-direction. Fluoroscopy is carried out by irradiating X-rays from the X-ray tube 216 to a patient placed on the pallet support 124 (or a patient pallet placed thereon), where X-ray fluoroscopy device 202 has been positioned at a given imaging position. X-rays transmitted by the patient during the irradiation are detected as an analog fluoroscopic image by the image intensifier and TV camera of the imaging unit 218.

In both FIGS. 1 and 2, the pallet support 124 (with a patient lying thereon or on a patient pallet thereon) enters and exits the bore 116 as the CT gantry is slid along its rails 112 to realize a given position within the gantry 110. The operation of CT scanners and the X-ray fluoroscopy device is well-known. See, for example, U.S. Pat. No. 5,995,581.

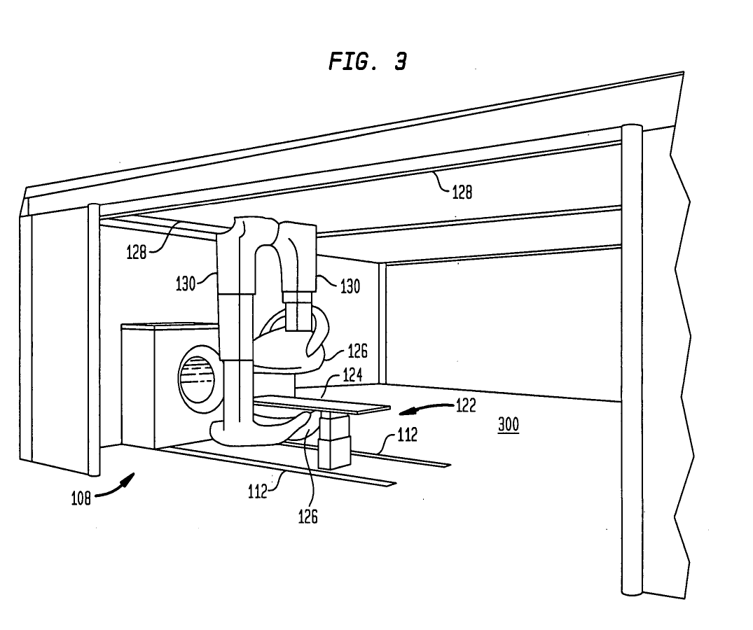

Referring now to FIG. 3, there is shown a pictorial representation of a diagnostic suite 300 showing the medical imaging apparatus 100 of FIG. 1. Here, the SPECT 126 is shown in position about the pallet support 124 in an in-use position.

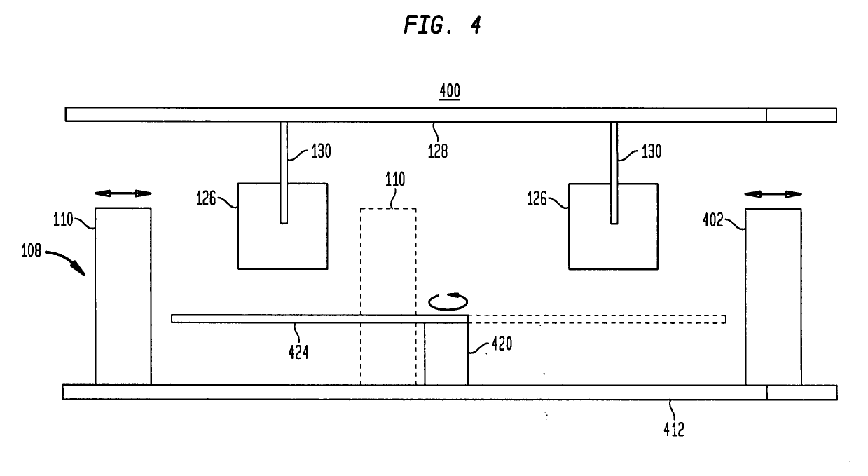

Referring now to FIG. 4, there is shown another embodiment of the medical imaging apparatus according to the present invention. The medical imaging apparatus 400 is like the medical imaging apparatus 100 in FIG. 1, with the exception that it also includes an another imaging device, for example. a sliding MRI 402. The sliding MRI 402 using the same tracks 412 as the CT device 108. An additional difference is the pallet support 424 is pivotally attached to the base 420. This way the end of the pallet support 424 that is not attached to the base 420 is pivoted to be parallel and pointing to the device 108 or 402 that is to be used and then secured in place in preparation for the next scan. In a scan episode where a CT and an MRI are performed, the pallet support 424 is pivoted while the patient is on it after the first scan is completed.

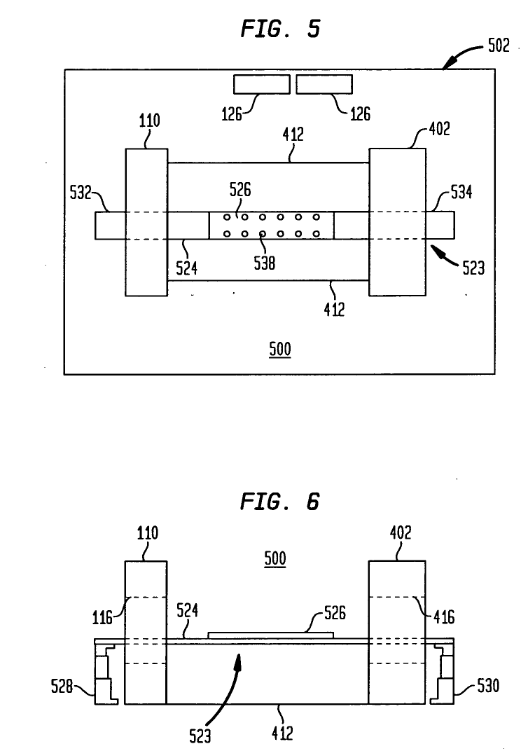

Referring now to FIGS. 5 and 6, there is shown another embodiment of the medical imaging apparatus and diagnostic suite according to the present invention. FIG. 5 is a plan view and FIG. 6 is an elevational view of the imaging apparatus according to this embodiment. The medical imaging apparatus 500 is contained in the diagnostic suite 502. The medical imaging apparatus 500 is like the medical imaging apparatus 400 in FIG. 4, with the exception that it uses a different pallet support structure 523. They are similar in that also includes a sliding MRI 402 using the same tracks 412 as the CT device 108. The SPECT 126 are also present. Focusing on the pallet support structure or table 523, it includes a table top structure 524 with a first end 532 and a second end 534 that extend through the bore 116 of the CT scanner 108 and the bore 416 of the MRI 402, respectively. The first end 532 has a first end support 528 that is vertically adjustable. The second end 534 has a second end support 530 that is vertically adjustable. The first and second end supports 528, 530 move the table top structure 524 vertically up and down to substantially align the patient support structure 523 with the respective bore 116, 416 of the first or second imaging device, respectively. A patient pallet 526 is placed on the table top structure 524 between the first and second bores 116, 416 of the first and second imaging devices when the first and second imaging devices 108 and 402 are in a non-use position.

The patient is secured to the patient pallet 526 and remains substantially immobile during both the CT and MRI scans. Preferably, the patient pallet 526 includes a series of fiducials or markers 538 disposed or mounted at known locations along the table top structure 524 (not shown) to aid in the registration of the resulting CT and MRI or other nuclear images. The markers can be imbedded within the table material or can be attached to the table in a fixed and stable manner. Optionally, fiducials can also be affixed to the patient.

The patient pallet 526 can be used with any of the embodiments shown in FIGS. 1 through 4.

The fiducials are constructed of a material(s) that is opaque to x-rays and that emits radiation in an energy band that is detected by the detector heads 126 of the SPECT. More particularly, the fiducials include radio nuclide point sources surrounded by or contained within a plastic sphere. The radio nuclide sources are visible in the resulting images and serve as an aid in accurate image registration and fusion, and as an aid in verification of accurate registration and fusion of the images from the scans of the various medical imaging devices.

However, the markers are preferably designed to have relatively weak source strengths and intermediate attenuation factors so that they image in the background but do not introduce artifacts or otherwise interfere with the image quality of the CT or the nuclear images. Thus, the markers can be refillable and thus filled when needed with relatively short lived radio nuclides such as Tc-99m, F-18, etc. Alternatively, the markers can be more permanent and contain longer lived radio nuclides such as Co-57, Gd-153, Ge-68, etc. Preferably, the fiducials include a further identification of individual fiducials.

In separate imaging episodes, body organs and tissue can move with respect to body bones over relatively short time intervals. Surgery and other trauma events are known to move anatomy within the body. Even subtle things such as the movement of gas, foods and liquids through the body can cause a relative shifting of the body's organs and tissues with respect to the body's frame—those shifts in position can confound accurate registration and fusion of multi-modality images. Also, different external conditions such as tables, table pads, sheets, patient clothing, etc. can cause significant problems with accurate image registration of multi-modality data sets. The subject system is designed to eliminate or at least minimize these confounding factors to accurate multi-modality image registration and fusion.

Thus, data and images acquired on the same table and in the same clinical room and environment and nearly at the same time (i.e. within about 30 minutes), are more valuable than comparable multi-modality images acquired at different times, and in a different imaging environment. The different imaging environment is intended to include the same patient in a different internal imaging state including position of organs, weight, disease or health state, etc. as well as a different external environment state including orientation, posture, clothes, table pads, sheets, room temperature, etc.

The invention has been described with reference to the preferred embodiment(s). Obviously, modifications and alterations will occur to others upon reading and understanding the preceding detailed description. It is intended that the invention be construed as including all such modifications and alterations insofar as they come within the scope of the appended claims or the equivalents thereof.

What is claimed is:

1. A multi-modality medical imaging apparatus comprising:
a first imaging device for obtaining one or more tomographic images of a subject patient, at least a portion of the first imaging device having a first bore;
a second imaging device for obtaining one or more tomographic images of a subject patient, at least a portion of the second imaging device having an imaging section;
a patient pallet;
a horizontally fixed patient support structure for supporting the patient pallet and having vertical adjustment to position the patient pallet in substantial alignment with the first bore of the first imaging device;
a first positioning device for translating the first imaging device axially along the patient pallet in forward and reverse directions during formation of one or more images by the first imaging device; and
a second positioning device for positioning the imaging section of the second imaging device relative to the patient support during formation of one or more images by the second imaging device.

2. The medical imaging apparatus of claim 1, wherein the imaging section of the second imaging device comprises a second bore.

3. The medical imaging apparatus of claim 2, wherein the patient support structure extends through the first and second bores of the first and second imaging devices, respectively, and the patient support structure comprises:
a table top structure with a first end and a second end,
wherein the first end includes a first end support that is vertically adjustable, and
the second end includes a second end support that is vertically adjustable, and
the first and second end supports move the table top structure vertically up and down to substantially align the patient support structure with the respective bores of the first or second imaging device,
wherein the patient pallet is located between the first and second bores of the first and second imaging devices when the first and second imaging devices are in a non-use position.

4. The medical imaging apparatus of claim 1, wherein the patient support structure comprises a vertically adjustable base and a cantilevered pallet support pivotally attached to the base, such that the pallet support in a first position is used with the first imaging device and in a second position is used with the second imaging device.

5. The medical imaging apparatus of claim 1, wherein the first imaging device is selected from the group consisting of CT, MRI, X-Ray, and Ultrasound devices.

6. The medical imaging apparatus of claim 5, wherein the second imaging device is different from the first imaging device and is selected from the group consisting of CT, MRI, X-Ray, and Ultrasound devices.

7. The medical imaging apparatus of claim 6, further comprising a third imaging device selected from the group consisting of SPECT and PET devices.

8. The medical imaging apparatus of claim 7, wherein the first imaging device comprises a sliding CT device, the second imaging device comprises a sliding MRI device, and the third imaging device comprises a SPECT device.

9. The medical imaging apparatus of claim 1, wherein the second imaging device is selected from the group consisting of SPECT and PET devices.

10. The medical imaging apparatus of claim 1, wherein the first imaging device comprises a sliding CT device and the second imaging device comprises a SPECT device.

11. The medical imaging apparatus of claim 1, wherein the patient pallet comprises a plurality of markers that are imaged by both the first imaging device and the second imaging device, wherein the plurality of markers are used in the preparation of a superimposed combined image representation.

12. A patient table connected to a medical imaging apparatus having a first imaging device with a first bore and a second imaging device with a second bore, comprising:
a patient support structure adapted to extend through the first and second bores of the first and second imaging devices, the patient support structure comprising:
a first end including a first end support that is vertically adjustable and
a second end including a second end support that is vertically adjustable,
wherein the first and second end supports move the patient support structure vertically up and down to substantially align the patient support structure with the respective bore of the first or second imaging device in an installed position; and
a pallet for supporting a patient on the patient support structure located between the first and second bores of the first and second imaging devices in an installed position.

13. A diagnostic imaging suite comprising:
a first medical imaging device for performing a first imaging procedure on a subject supported on a patient pallet, wherein the first medical imaging device has a first sliding gantry;
a second medical imaging device positioned separately from the first medical imaging device for performing a second imaging procedure on said subject;
a patient pallet support having vertical adjustment for adjusting the vertical positioning of said patient pallet; and
a pair of rails positioned parallel to a longitudinal axis of the patient pallet support, each of the rails being positioned on an opposite side of the patient pallet support for use with the first sliding gantry;
wherein the second imaging system is positionable in a rest position and in an in-use position, such that in the rest position the second imaging system does not interfere with the first imaging system when in use.

14. The diagnostic imaging suite of claim 13, wherein the second medical imaging device has a second sliding gantry.

15. The diagnostic imaging suite of claim 14, wherein the first and second gantry both use the pair of rails.

16. A multi-modality medical imaging apparatus, comprising:
a stationary patient table;
a first imaging device of a first modality movably supported for motion with respect to said stationary patient table; and
a second imaging device of a second modality different than said first modality, movably supported for motion with respect to said stationary patient table.
